(19) United States
(12) Patent Application Publication
(10) Pub. No.: US 2005/0010263 A1
(43) Pub. Date: Jan. 13, 2005

(54) Neurostimulation unit for immobilizing the heart during cardiosurgical operations

(76) Inventor: Patrick Schauerte, Aachen (DE)

Correspondence Address: OHLANDT, GREELEY, RUGGIERO & PERLE, LLP, ONE LANDMARK SQUARE, 10TH FLOOR, STAMFORD, CT, 06901, US

(21) Appl. No.: 10/485,040

(22) PCT Filed: Jul. 29, 2002
(86) PCT No.: PCT/EP02/08443

(30) Foreign Application Priority Data
Jul. 27, 2001 (DE) 10136567.5

(51) Int. Cl. A61N 1/18

(52) U.S. Cl. 607/48

(57) ABSTRACT

The invention relates to a device for temporary reduction of heart movement during an operation, more particularly a heart operation, comprising a neurostimulation unit (20) for stimulation of the nerves (3) that slow down heart frequency, said unit including at least one electrode device (1) with at least one stimulation pole (2), wherein a control unit (19) connected to the neurostimulation device (20) is provided. Said control device has a first input device (26) for inputting a degree of immobilization and is configured to influence the operating state of the neurostimulation device (20) depending on the previously set degree of immobilization.

This invention relates to a stimulation device with which it is possible to selectively electrically stimulate the epicardial or extracardiac parasympathetic nerves of the heart innervating the sinus node or the atrioventricular node to electrically largely immobilize the heart during cardiac surgery by slowing the heart rate.

Cardiovascular support during cardiac surgery by means of an extracorporeal circulation of a heart-lung machine offers specific disadvantages. Critical factors include in particular the incidence of systemic inflammations and immune reactions, the thrombogenic effect of foreign body material, the reduced cardiac output especially due to the potassium cardioplegia (low-output state) and the altered flow dynamics under the conditions of artificial circulation (Cremer et al. Ann. Thorac. Surg. 1966; 61: 1714-20; Myles et al. Med. J. Austr. 1993; 158: 675-7; Roach et al. N. Engl. J. Med; 335: 1857-63; G. M. McKhan et al. Ann. Thorac. Surg. 1997; 63: 516-521; Ede et al. Ann. Thorac. Surg. 1997; 63: 721-7). The reduced perfusion to all organs of the body, in particular during surgery, which is influenced by many of these factors, may lead to permanent organ damage such as neurophysiological and neuro-psychological damage, even including a stroke or renal failure, for example (Cremer et al. Ann. Thorac. Surg. 1966; 61:1714-20; Myles et al. Med. J. Austr. 1993; 158:675-7; Roach et al. N. Engl. J. Med. 335:1857-63; G. M. McKhan et al. Ann. Thorac. Surg. 1997; 63:516-521).

Therefore, methods have been developed for creating a bypass to the coronary vessels without requiring cardiovascular support by extracorporeal circulation using the heart-lung machine during heart surgery.

Local stabilizers of the myocardial area in the immediate vicinity of a coronary vessel make it possible to create a coronary anastomosis on a beating heart, for example (Boonstra et al. Ann. Thorac. Surg. 1997; 63:567-9).

Heart surgery with circulatory support by microaxial pumps is another gentle alternative to surgery with the assistance of a heart-lung machine. Microaxial pumps for cardiac support have been known for a long time. They have a flywheel which supports the blood flow and is frequently referred to as a rotor or impella and rotates about an axis situated coaxially in the blood vessel. European Patent EP 0 157 871 B1 and European Patent EP 0 397 668 B1 describe a microaxial pump in which the flywheel is connected to an extracorporeal drive part via a flexible shaft running through the catheter. The drive part drives the flexible shaft, which in turn drives the flywheel of the microaxial pump.

Recent developments in microaxial pumps, which are already in use clinically, involve combining the drive part and the pump part in one unit and implanting them as a unit in the vascular system of the body. Instead of the mechanically susceptible drive shaft, only a power supply cable for supplying electric power to the drive part passes through the catheter. Such a microaxial pump, also known in general as an “intravascular blood pump” is described in German Patent DE 196 13 564 C1, for example.

A pump system comprising two pumps may be used to assume all or part of the pumping function of the heart (described in PCT/EP/98/01868). Such a system is capable of handling a cardiac output of approximately 4.5 liters of blood per minute and can reduce an increased wall excursion during bradycardia, for example. The pump device has a first pump, which may be inserted with its intake side into the left ventricle, while a second pump, which is situated with its intake side in the right atrium, lies with its pressure side in the pulmonary artery. The two pumps are operated by a shared control unit. The two pumps are introduced into the heart without having to open the ventricle.

A so-called “paracardial blood pump” (German Patent Application 100 16 422.6-35) is also able to handle an even higher cardiac output of five to six liters of blood per minute, and, thus, to minimize the increased wall excursion which occurs in bradycardia due to the fact that heart function is completely taken over by the pump. In contrast with the “intravascular pump” described above, this is a blood pump which draws in blood from one part of the heart and delivers it into the aorta or some other target region, the casing of the blood pump being applied to the outside of the heart while the pump inlet has a direct connection to the chamber of the heart from which the blood is drawn.

If the entire cardiac output is handled during a severe episode of bradycardia by using the intra- or paracardial pump described above, the fluid balance is established by the sensors integrated into the pumps, allowing monitoring of the blood flow delivered in a mutual dependency of the pumps.

In contrast with surgery using the heart-lung machine, the heart still continues to beat due to the persistence of its own electric activity—despite the fact that the cardiac output is being handled by the blood pump—but it does so without pumping any relevant blood volume. This mechanical action makes open-heart surgery difficult and increases the myocardial oxygen consumption.

To minimize this movement of the heart during surgery, a stimulation device according to the present invention is described below with which it is possible to selectively electrically stimulate the epicardial or extracardiac parasympathetic nerves of the heart innervating the sinus node or the atrioventricular node to largely electrically immobilize the heart during heart surgery by reducing the heart rate. In particular, the combination of such a neurostimulation unit with intracardial or paracardial blood pumps is described to ensure perfusion of organs, including the heart, during bradycardia.

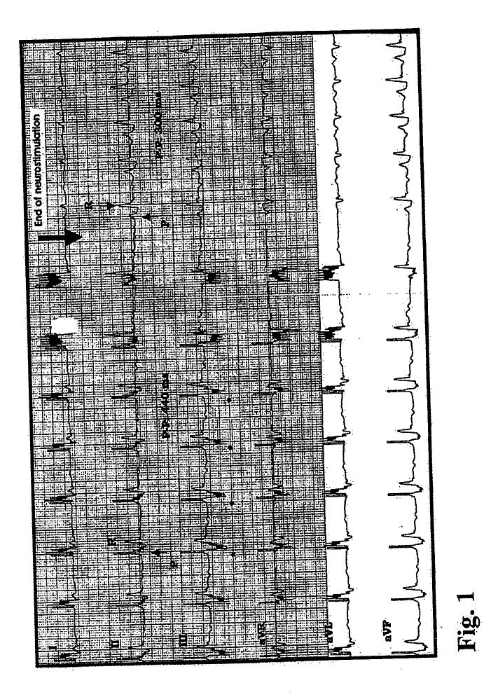

On the healthy heart, the spontaneous heart rate is determined by the pulse generation rate of the pacemaker center of the heart, the so-called sinus node. The sinus node is located on the high lateral right atrium. The electric conduction of the stimulation of the atria to the chambers of the heart is in turn accomplished via the so-called atrioventricular (AV) node. The vegetative autonomic nervous system consists of a stimulating part, the sympathetic nervous system, and a sedative part, the parasympathetic nervous system. Activation of the parasympathetic nervous system causes the sinus node frequency to be slowed down (negative chronotropic effect) and leads to a delay in the atrioventricular conduction via the AV node (negative dromotropic effect). Parasympathetic nerves innervating the sinus node and the AV node extracardially run along the superior vena cava and along the pulmonary arteries to the sinus node or AV node and then cluster near the target organ in circumscribed epicardial accumulations of fat and connective tissue (so called nerve plexus or “fat pads”). The nerve plexus, which contains almost all the parasympathetic fibers that innervate the sinus node, is situated epicardially on the lateral right atrium in a corner between the right atrial wall and the right pulmonary veins crossing behind the right atrial wall (so-called ventral right atrial plexus). The nerve plexus which contains most of the parasympathetic nerve fibers innervating the atrioventricular node is situated in a corner between the coronary sinus ostium, the inferior vena cava and the left atrium (the so-called inferior inter-atrial plexus). FIG. 1 shows a schematic representation of these parasympathetic nerve plexus.

The epicardial electric stimulation of the right ventral atrial plexus triggers a sinus bradycardia without having any relevant influence on the AV node conduction. Epicardial or transvascular electric stimulation of the inferior inter-atrial plexus slows down the atrio-ventricular conduction (P. Schauerte et al. Catheter stimulation of cardiac parasympathetic nerves in humans. A novel approach to the cardiac autonomic nervous system. Circulation, 2001; 104: 2430-2435) but has no effect on the sinus node frequency. Epicardial or transvascular stimulation of the two nerve plexus leads to shortening of the local atrial refractory time in the vicinity of the respective nerve plexus and can result in a slight reduction in the atrial contractility. However, the ventricular pumping force or refractory time is not influenced significantly. The transvascular nerve stimulation thresholds are much higher than the epicardial stimulation thresholds (P. Schauerte et al. Ventricular Rate Control During Atrial Fibrillation by Cardiac Parasympathetic Nerve Stimulation. A Transvenous Approach. J. Am. Coll. Cardiol. 1999; 34: 2043-2050). Parasympathetic fibers innervating the sinus node and the AV node may also be stimulated electrically extravascularly or transvascularly along/in the vena cava, which also leads to negative chronotropic and dromotropic effects (P. Schauerte et al. Ventricular Rate Control During Atrial Fibrillation by Cardiac Parasympathetic Nerve Stimulation. A Transvenous Approach. J. Am. Coll. Cardiol. 1999; 34: 2043-2050; P. Schauerte et al. Transvenous parasympathetic nerve stimulation in the inferior vena cava and atrioventricular conduction. J. Cardiovasc. Electro-physiol. 2000; 11: 64-69; P. Schauerte et al. Transvenous parasympathetic cardiac nerve stimulation: An approach for stable rate control. J. Cardiovasc Electrophysiol. 1999; 10: 1517-1524; P. Schauerte et al. Treatment of tachycardiac atrial fibrillation by catheter-supported electric stimulation of the cardiac parasympathetic nervous system. Z Kardiol. 2000; 89: 766-773; P. Schauerte et al. Transvascular radiofrequency current catheter ablation of parasympathetic cardiac nerves abolishes vagally mediated atrial fibrillation. Circulation, 2000; 28: 2774-2780).

In addition, this results in shortening of the atrial refractory time but an extension of the ventricular refractory time and a slight reduction in the atrial contractility. Parasympathetic fibers innervating the sinus node and the AV node may also be stimulated along the right or left pulmonary artery, which also causes negative chronotropic and dromotropic effects. The parasympathetic fibers along the superior vena cava and along the pulmonary arteries are preganglionic nerve fibers while both preganglionic and postganglionic nerve fibers accumulate in the inferior inter-atrial plexus (P. Schauerte et al. Transvascular radiofrequency current catheter ablation of parasympathetic cardiac nerves abolishes vagally mediated atrial fibrillation, Circulation, 2000; 28: 2774-2780).

FIG. 1 illustrates the effect of electric stimulation of the inferior interatrial plexus. The frequency-retarding effect is instantaneous, i.e., it begins immediately with the onset of nerve stimulation and terminates immediately after the end of stimulation. In addition, it is “titratable,” i.e., the extent to which the heart rate is slowed down can be adjusted through the choice of the corresponding stimulation voltage.

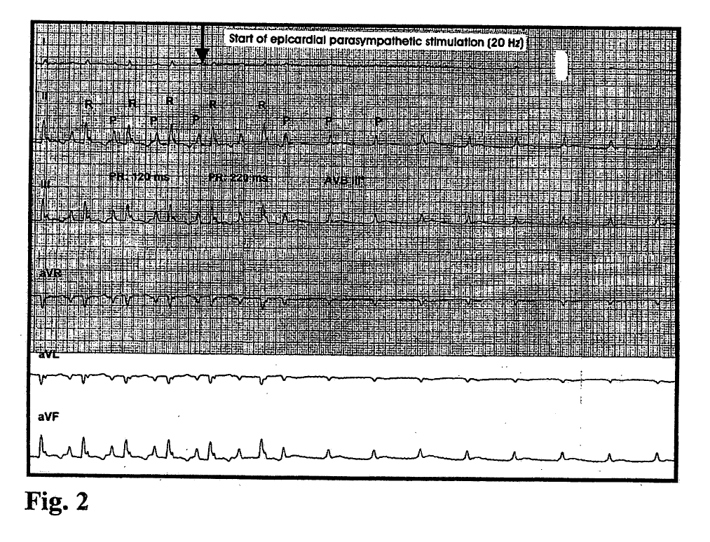

FIG. 2 shows an example of parasympathetic stimulation of the ventral right atrial plexus.

The object of the present invention is to create a device which will temporarily reduce the heart rate or stop the heart from beating by transient intraoperative epicardial or transvascular electric parasympathetic stimulation in order to facilitate, by this temporary electric reduction in heart movement, the job of the surgeon/robot in guiding the surgical instruments at the heart.

This object is achieved with the invention by a device for temporarily reducing the movement of the heart during surgery, in particular during cardiac surgery, with a neurostimulation unit for stimulating nerves that slow down the heart rate, comprising at least one electrode device having at least one stimulation electrode. According to the present invention, a control unit is connected to the neurostimulation unit, said control unit having a first input device for preselecting of a degree of immobilization and being arranged for influencing the operating mode of the neurostimulation unit as a function of the predetermined degree of immobilization.

Any bradycardia is normally associated with an increase in stroke volume. Therefore, intraoperative bradycardia would reduce the number of contractions per minute, but a single contraction would lead to a greater inward-outward movement of the wall of the heart, which would counteract immobilization of the heart. In other words, frequent slight wall excursions without neural stimulation would be replaced by a few major wall movements during neural stimulation.

To compensate for this disadvantage of bradycardization, this invention, in an advantageous modification provides for a combination of the neurostimulator with another movement-reducing device also being connected to the control unit and having the form of an intravascular/intracardiac pump which takes over a portion of the mechanical pumping function of the heart. The cardiac output taken over by the pump can be adapted at any time to the extent of the bradycardia, i.e., the greater the bradycardia, the greater is the cardiac output transported through the pump. In this way an adequate cardiac output is maintained during bradycardia, but at the same time the increased stroke volume and consequently the increased wall excursion of bradycardia are reduced, which results in a more effective immobilization of the heart than that obtained with an intravascular pump or a nerve stimulation alone.

The combination of a neurostimulation unit with a movement reducing device in the form of a stabilization device for stabilizing the cardiac wall, e.g., a local stabilization/immobilization device, is provided according to the present invention. The bradycardia achieved with the neurostimulation unit and the electric immobilization should cooperate additively to the local immobilization achieved through the local stabilization systems.

Other preferred embodiments of this invention are derived from the dependent claims and/or the description of preferred embodiments given below and referring to the accompanying drawings. It is shown in:

FIG. 1 an electrocardiogram at electric stimulation of the inferior interatrial plexus;

FIG. 2 an electrocardiogram at parasympathetic stimulation of the ventral right atrial plexus;

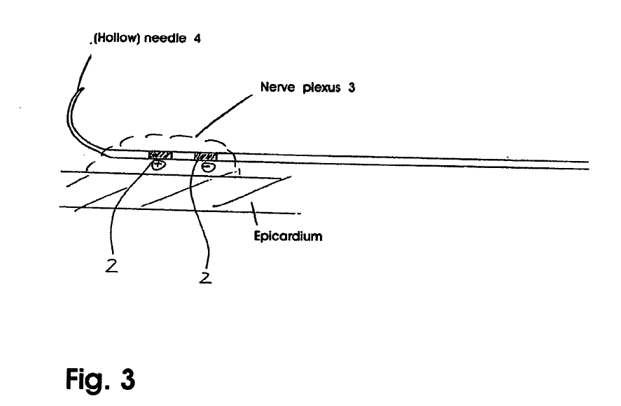

FIG. 3 a preferred embodiment of an electrode device of an apparatus according to the present invention;

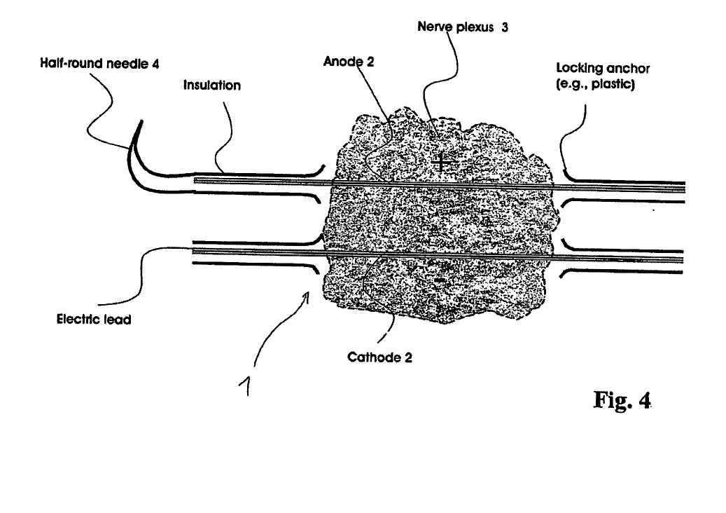

FIG. 4 another preferred embodiment of an electrode device of an apparatus according to the present invention;

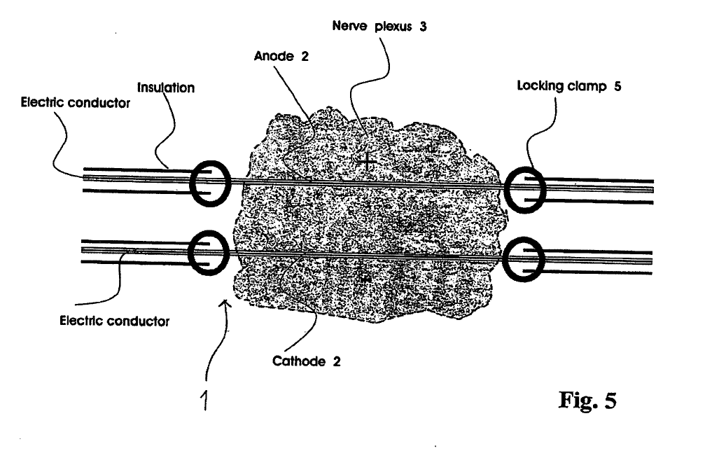

FIG. 5 another preferred embodiment of an electrode device of an apparatus according to the present invention;

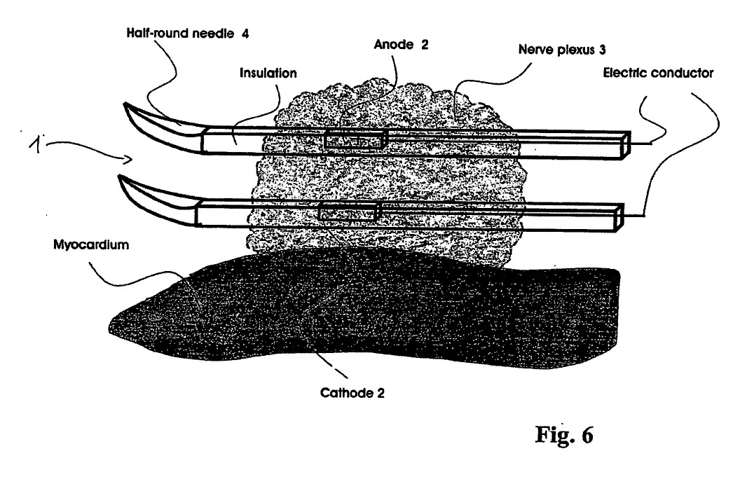

FIG. 6 another preferred embodiment of an electrode device of an apparatus according to the present invention;

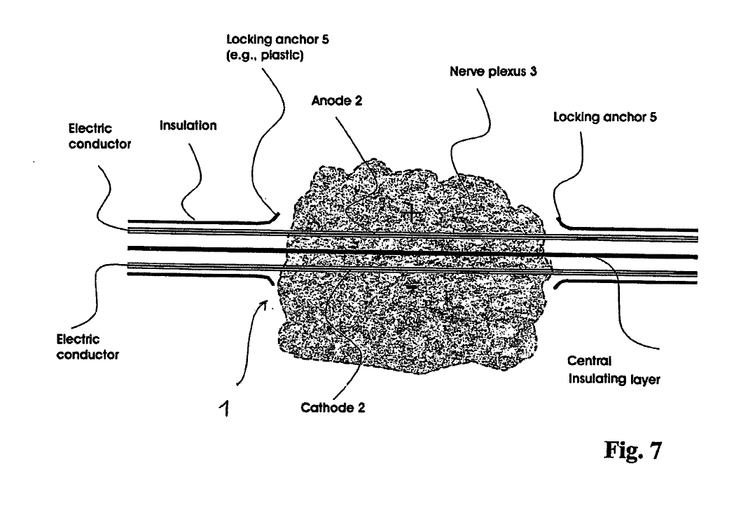

FIG. 7 another preferred embodiment of an electrode device of an apparatus according to the present invention;

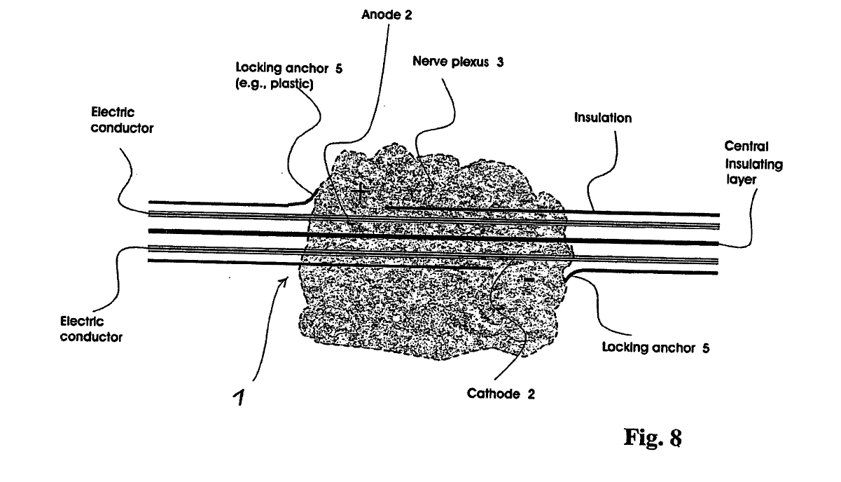

FIG. 8 another preferred embodiment of an electrode device of an apparatus according to the present invention;

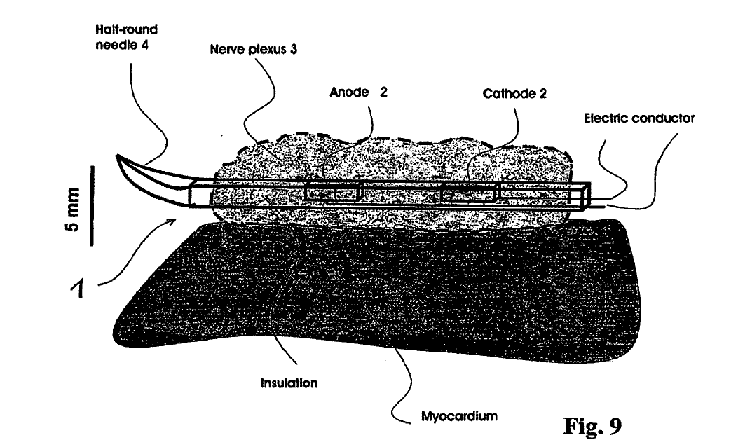

FIG. 9 another preferred embodiment of an electrode device of an apparatus according to the present invention;

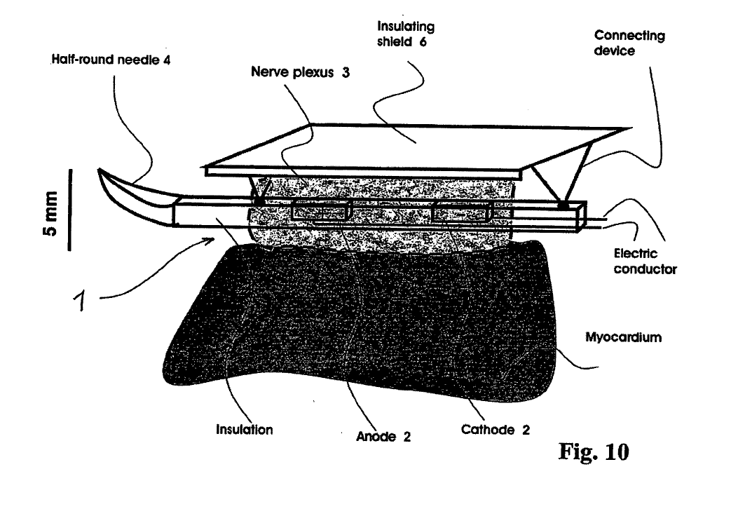

FIG. 10 another preferred embodiment of an electrode device of an apparatus according to the present invention;

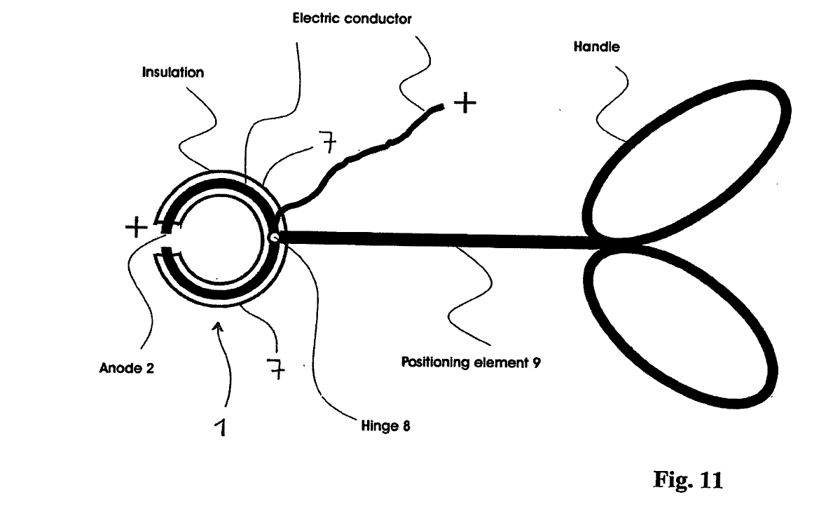

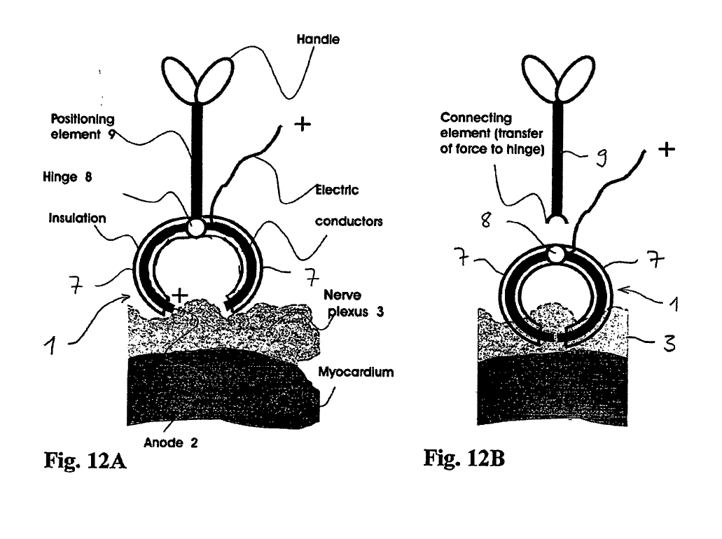

FIG. 11 another preferred embodiment of an electrode device of an apparatus according to the present invention;

FIG. 12 another preferred embodiment of an electrode device of an apparatus according to the present invention;

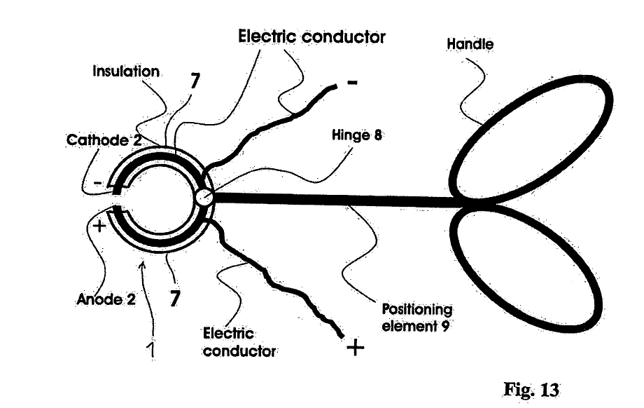

FIG. 13 another preferred embodiment of an electrode device of an apparatus according to the present invention;

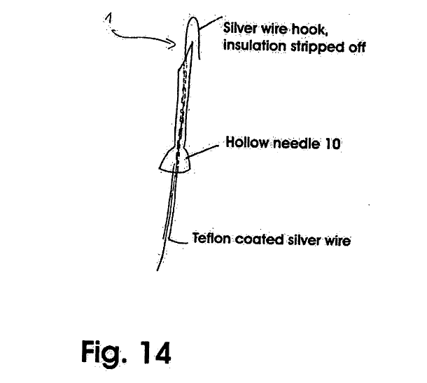

FIG. 14 another preferred embodiment of an electrode device of an apparatus according to the present invention;

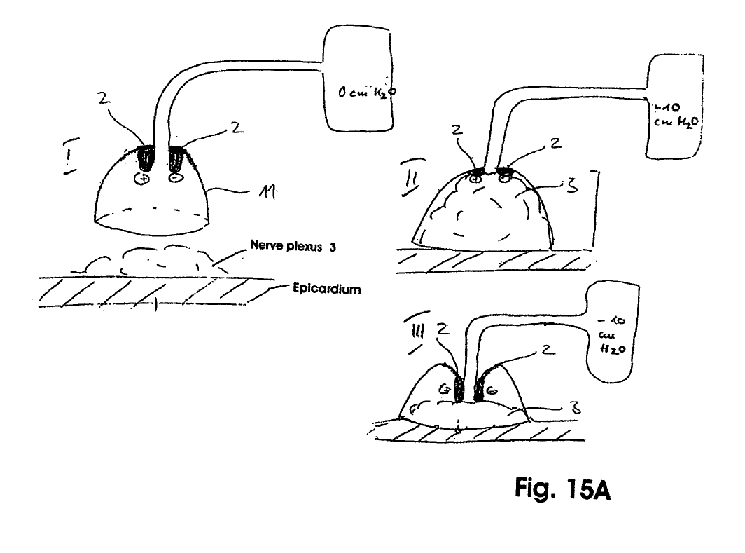

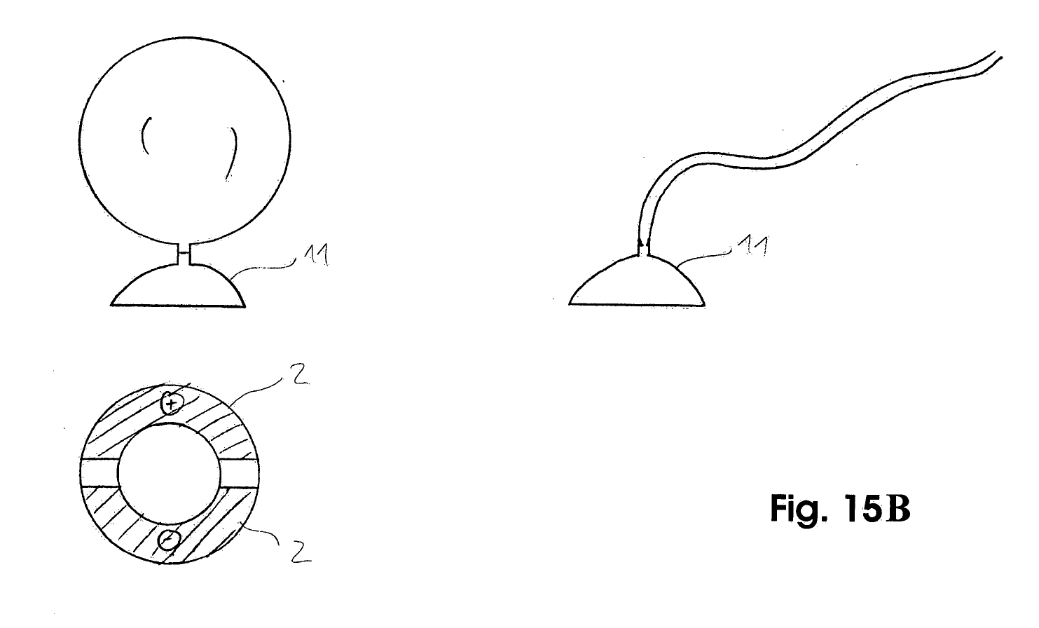

FIGS. 15A and 15B another preferred embodiment of an electrode device of an apparatus according to the present invention;

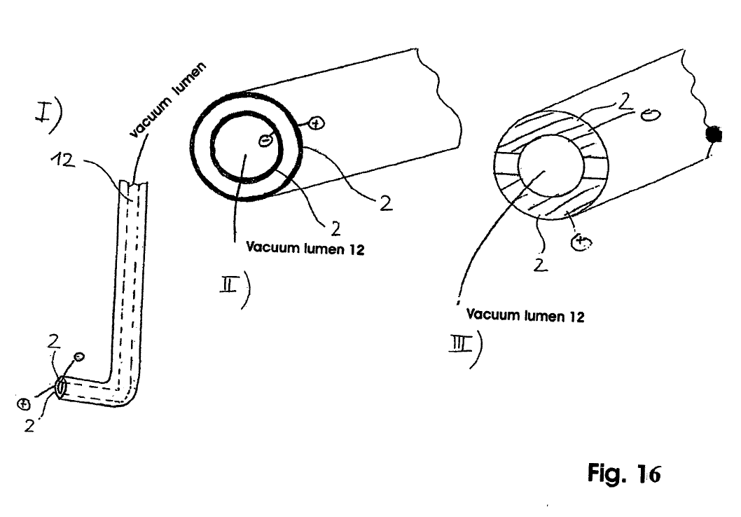

FIG. 16 another preferred embodiment of an electrode device of an apparatus according to the present invention;

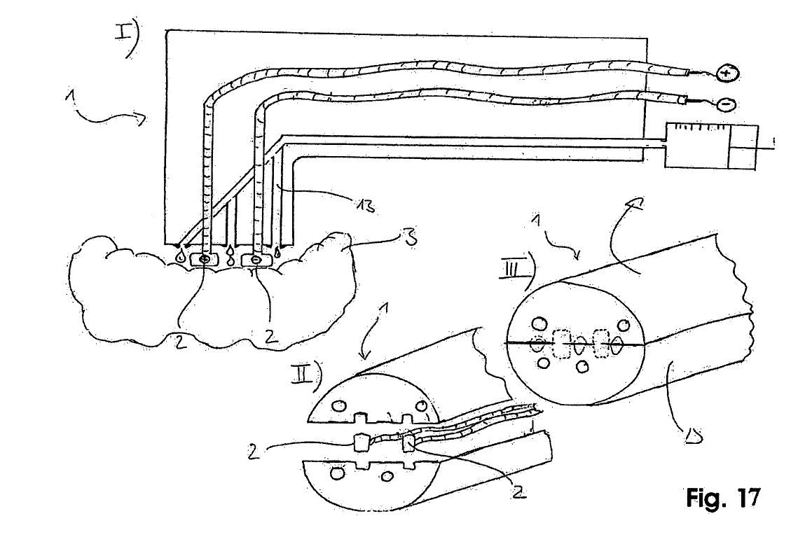

FIG. 17 another preferred embodiment of an electrode device of an apparatus according to the present invention;

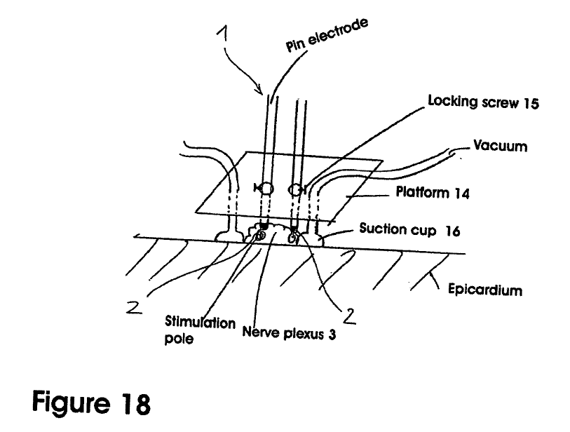

FIG. 18 another preferred embodiment of an electrode device of an apparatus according to the present invention;

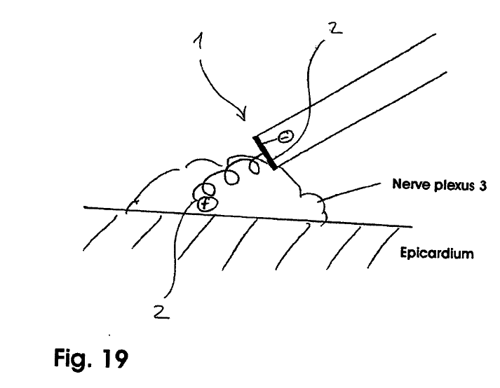

FIG. 19 another preferred embodiment of an electrode device of an apparatus according to the present invention;

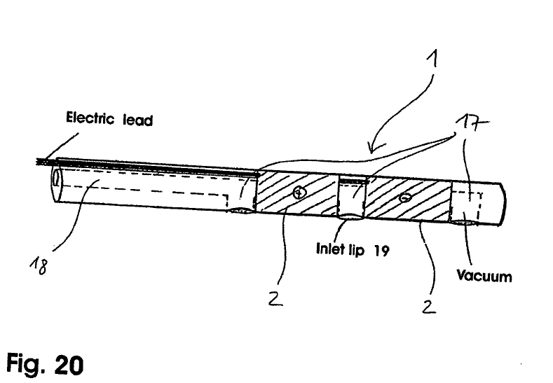

FIG. 20 another preferred embodiment of an electrode device of an apparatus according to the present invention;

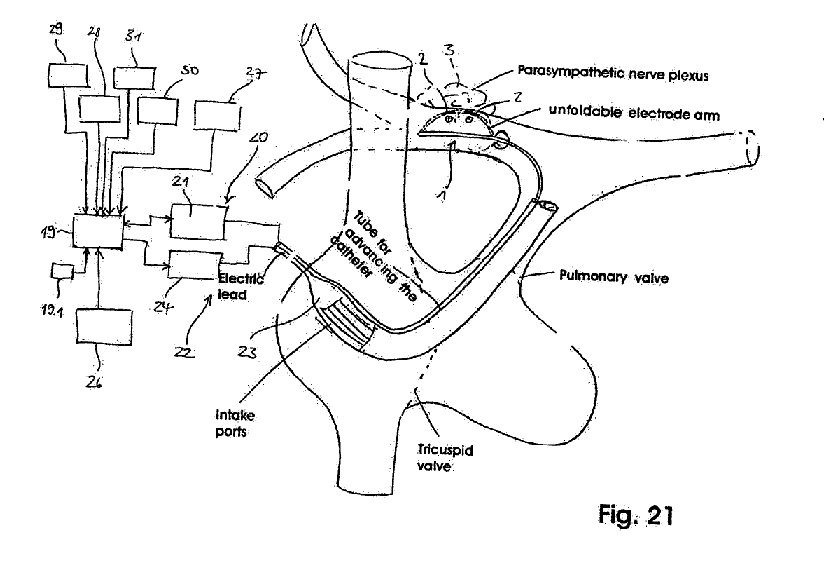

FIG. 21 another preferred embodiment of an apparatus according to the present invention;

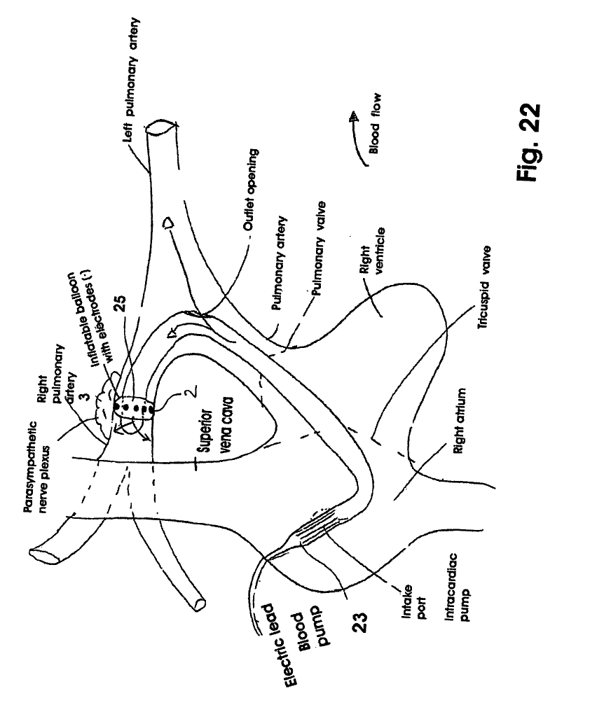

FIG. 22 another preferred embodiment of an apparatus according to the present invention;

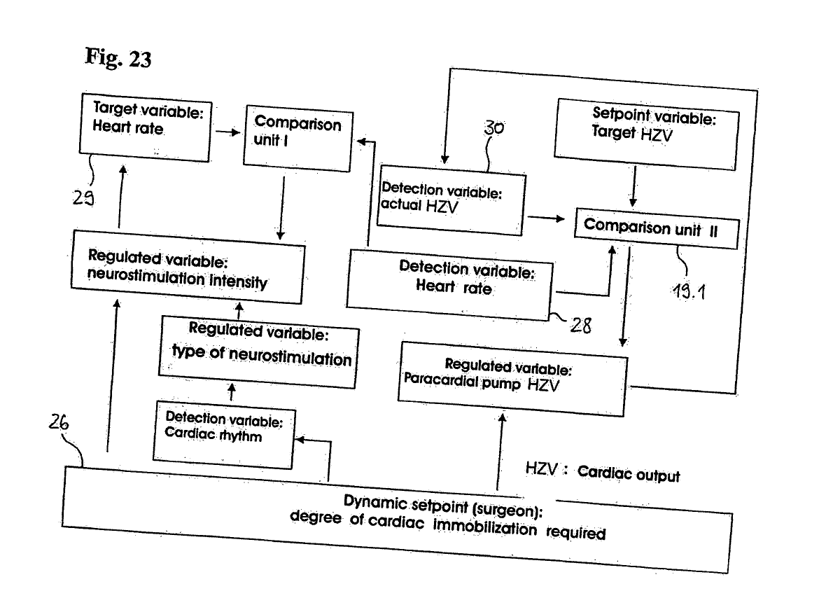

FIG. 23 a block schematic of the control loop of another preferred embodiment of the apparatus according to the present invention;

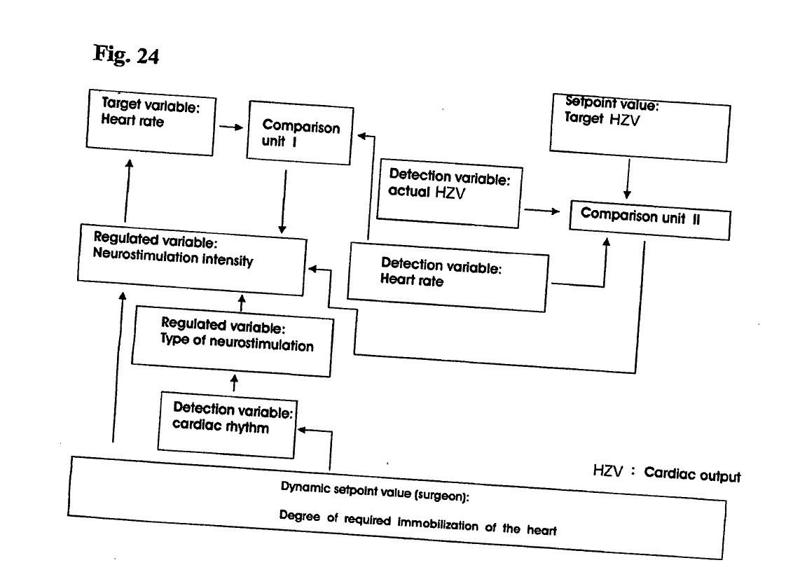

FIG. 24 a block schematic of the control loop of another preferred embodiment of the apparatus according to the present invention.

FIG. 1 shows an example of the parasympathetic stimulation of the ventral right atrial plexus with consecutive sinus bradycardia (P-P interval 440 ms). To prevent atrial myocardial stimulation, the high-frequency (200 Hz) nerve stimuli (*) were triggered in the atrial refractory time. Immediately after the end of neurostimulation (thick vertical arrow) in the atrial refractory time, there is again a rise in the sinus node frequency (P-P interval 300 ms). R denotes the R wave and P denotes the P wave.

FIG. 2 shows the effect of epicardial electric stimulation of the inferior right atrial plexus in a dog. The frequency retarding effect is instantaneous, i.e., it starts immediately with the onset of neurostimulation and terminates immediately after neurostimulation is stopped. Here again, R denotes the R wave and P denotes the P wave.

According to the present invention, the neurostimulation unit comprises an electrode device in the form of a stimulation electrode 1 which is attached epicardially to the ventral right atrial plexus, the inferior interatrial plexus, the superior vena cava or the right or left pulmonary artery. The introduction of such an electrode may be performed in the open thorax after performing a thoracotomy. As an alternative, however, the neurostimulation electrodes may also be placed by trocar at the stimulation sites endoscopically/robot controlled. In a typical embodiment, the electrode has one or two electrically conducting stimulation poles 2.1, whose effective stimulation area amounts to between 1 and 100 mm² (preferred embodiment, 4-9 mm²) (see FIGS. 3-7). The stimulation poles may be part of an electric conductor (stimulation wire) insulated with plastic, the insulation being stripped off of said conductor in the area of the stimulation pole 2. The stimulation wire is pulled through the epicardial nerve plexus 3 so that the stimulation pole 2 comes to lie within the nerve plexus. To facilitate insertion into the nerve plexus 3, one embodiment of the stimulation wire has a tapered point or needle 4, making it possible to puncture through the nerve plexus 3. In a typical embodiment, this is a (half)-round needle 4 which makes it possible to puncture through the epicardial nerve plexus 3 at the surface. To permit bipolar stimulation of the nerve plexus, two stimulation wires are placed in the nerve plexus 3 spaced a distance of approx. 2-10 mm apart. Alternatively, two mutually insulated electric conductors may be combined in a shared stimulation electrode (see FIGS. 6-8). Each of the two insulated conductors has a stimulation pole 2 at different distances from the electrode tip 4.

To prevent dislocation of the stimulation electrode 1 out of the nerve plexus 3, in a typical embodiment there is a locking device 5 on both sides of the stimulation electrode 1. These may be, for example, two plastic anchors on the two sides of the stimulation electrode 1 (see FIGS. 3, 6, 7) or a clamp which is attached to both sides after positioning the electrode (see FIG. 4).

To prevent, above all, myocardial stimulation of the adjacent or superior ventricular myocardium especially when the inferior interatrial plexus is stimulated, in a particular embodiment, a shielding device in the form of an insulating cap 6, which is made of a material that is not electrically conductive, may be temporarily attached to both sides of the stimulation electrode 1 with a locking device 5, so that the stimulation poles 2 located within the plexus 3 and the plexus 3 are insulated from the surrounding/superior myocardium (see FIG. 9). Alternatively, the stimulation poles 2, which are mounted on a stimulation electrode 1 designed to be flat, may be electrically shielded on one side (see FIG. 8). This makes it possible to position the stimulation electrode 1 with the electrically conducting surface/side facing the epicardium and provide a shield 6 with respect to the (ventricular) myocardium above it. Likewise, the stimulation electrode 1 may also be positioned with the electrically conducting surface/side facing away from the epicardium so that the insulated surface 6 is in contact with the epicardium. This results in the electrode 1 being positioned between the epicardium and the plexus 3 above it, so that simultaneous (atrial) myocardial stimulation during neurostimulation is prevented. In another embodiment, a stimulation electrode 1, which is designed to be flat, may also be combined with an insulation cap 6. In this case, however, the electrically conducting surface of the electrode 1 is placed, in the plexus 3, facing away from the epicardium and the insulating cap 6 is attached to the stimulation electrode with a locking device on both sides of the stimulation electrode (see FIG. 9). This may prevent electric stimulation of the (atrial) myocardium which is beneath the plexus 3 as well as the ventricular myocardium above it.

In another embodiment, the stimulation electrode consists of a ring electrode 1 which is composed of two half-round arms 7 (FIGS. 10-12). The proximal ends are movably secured with the distal ends being in contact end-to-end or overlapping at the ends. The distal ends of both semicircles can be pulled apart by a push/pull mechanism acting on the hinge 8 of the semicircles and being transmitted through a positioning element 9 and said distal ends behave like two forceps arms 7 which can grip the tissue of the nerve plexus 3 (see FIG. 12A). Because of the elastic restoring forces of the two semicircles or due to a renewed force acting on the hinge 8, the arms 7 of the circle then close and are thus secured in the nerve plexus 3. The arms 7 of the forceps therefore act like a fastening element for securing the ring electrode 1. The positioning element 9 is then removed from the hinge 8 (see FIG. 12B). The two semicircles 7 are made of an electrically conducting material and are coated with a non-electrically conducting substance except for the distal ends. The semicircles 7 are connected to a flexible electric conductor that is electrically insulated toward the outside. By placing two such ring electrodes 1 in a nerve plexus 3, bipolar stimulation is possible. According to one variant of this embodiment, two opposite electric poles 2 are arranged on one ring electrode 1 (see FIG. 13).

An alternative embodiment of the stimulation electrode 1 consists of a thin flexible silver wire coated with Teflon, for example, with the insulation being removed from the tip of the silver wire for a length of approximately 5-10 mm. This wire can be inserted through a traditional hollow needle 10 made of steel such as those used for venous vascular puncture (e.g., 20 gauge needle) (FIG. 14). As soon as the wire protrudes by approximately 5 mm out of the tip of the hollow needle, the tip of the wire is bent over to form a hook at its point of departure from the hollow needle. The hollow needle 10, which may also be designed as a round needle, is then inserted into the nerve plexus 3 (“fat pad”) so that the wire hook from which the insulation has been stripped comes to lie within the nerve plexus. Then the needle 10 is cautiously retracted, so that the wire hook remains in the nerve plexus. For bipolar electric stimulation of the nerve plexus, two of these Teflon-coated stimulation wires 1 with the stimulation poles 2 are placed each within a nerve plexus 3. The distance between the two stimulation poles 2 should be between 1 and 10 mm.

In a modified embodiment, the stimulation electrodes are incorporated into a intake device in the form of a suction bell or suction tube 11 to which is applied a permanent vacuum to reliably secure the stimulation poles 2 epicardially on the nerve plexus 3 or extravascularly on vessels (see FIG. 15). The suction bell 11 is in the shape of a hemisphere. The largest diameter of the hemisphere is 5-15 mm with a typical diameter being 5 mm. In a preferred variant, the suction bell 11 is made of plastic. An inlet opening provided on the suction bell 11 which is connected to a suction tubing through which a vacuum can be applied. The vacuum can be applied through an external suction via a tubing or through a local vacuum reservoir. Such a local vacuum reservoir could be, for example, a small rubber ball equipped with an outlet valve and connected to the inlet opening of the suction bell 11. When manual compression is applied to the rubber ball, air escapes through the outlet valve when the suction bell 11 is at the same time placed on the nerve plexus 3 and/or the blood vessel. After the compression is eliminated, the elastic restoring forces of the balloon create a vacuum which pulls the nerve plexus 3 into the suction bell 11 and thus results in the suction bell 11 and the stimulation poles 2 being secured on the nerve plexus 3 or the vessel, respectively.

On the inside of the suction bell, next to the inlet opening are provided two metal stimulation poles which are connected to thin electric conductors secured along the suction tubing. The suction bell 11 is placed on the ventral/inferior interatrial plexus/superior vena cava/right or left pulmonary artery while applying a vacuum. The vacuum causes the fatty tissue and nerve tissue to be sucked into the hemisphere so that it comes in contact with the stimulation poles 2. To prevent dislocation of the suction electrode, e.g., in luxation of the heart out of the pericardial sac, the vacuum may be increased briefly. According to an alternative embodiment, two stimulation electrodes are provided on the contact surface of the suction bell 11, coming in contact with the epicardial nerve plexus 3 in the area of the circumference of the suction bell 11. In both embodiments, the contact surface may be planar, concave or convex to ensure a tight closure of the suction bell 11 with the myocardium/nerve plexus 2.

An intake device in the form of a suction tube with a central vacuum lumen 12 (e.g., in the form of a hockey stick to reach the inferior interatrial plexus 3) and two electrodes 2 on the head side may be used for epicardial neurostimulation and are also within the scope of this invention (see FIG. 16).

According to an alternative variant, epicardial stimulation poles are secured by using a fibrin adhesive injection gun (see FIG. 17). The glue gun consists of two half tubes which together form a tightly sealed round tube in the form of a hockey stick. At the head end of the short arm of this tube two round holding fixtures have been secured, these in turn holding two metal pins at each distal end of which (the end outside of the tube) is mounted a disk-shaped stimulation pole 2. The electric conductors are mounted on the proximal ends of the pins and come out of the tube again at the head end of the long tube arm. First, the assembled tube having the stimulation poles 2 coming out at the head part of the short tube arm is used as a manually guided stimulation electrode 1 to identify the effective stimulation pole by probatory electric stimulation. After discovering an effective stimulation point, the stimulation pole 2 is attached. To do so, a plurality of openings are provided on the head end of the short tube arm in addition to the pin holding bars, said openings each being connected to a small reservoir at the long tube end via inlet channels in the form of tubular elements 13. Then, using a syringe connected to the outside of the reservoir, fibrin adhesive, for example, can be injected through the tubular elements 13 and the mouth openings on the head part of the short arm of the tube, so that the stimulation poles 2 positioned on the nerve plexus 3 are completely surrounded by fibrin adhesive. After brief hardening of the fibrin adhesive, the half tubes are opened up and removed so that the stimulation poles 2 together with the pins and the attached electric conductors are “welded” to the nerve plexus 3 by the fibrin adhesive. This embodiment is particularly suitable for attaching stimulation poles 2 to neurostimulation sites that are difficult to access such as the inferior interatrial plexus or the nerve plexus between the right pulmonary artery and the base of the aorta as well as the superior vena cava.

According to another variant, a small platform 14 is provided, having two or more holes provided in it through which the two stimulation poles 2 on pins 1 can be pushed through (see FIG. 18). A screw 15 at or above the inlet opening allows the electrode pin 1 to be attached so that different lengths of the electrode pin 1 beneath the platform 14 can be achieved. The platform 14 itself has two or more intake devices in the form of suction cups 16 with which it is positioned on the epicardial nerve plexus 3. After applying a vacuum to the suction cups 16, the platform is stabilized and secured above the nerve plexus 3. The stimulation pins 1 are then pushed through the inlet openings to the extent that they come in contact with the nerve plexus 3 and then are secured in this position by the locking screws on the platform 14.

According to another embodiment variant, a screw electrode 1 is anchored in the epicardial nerve plexus 3. The screw electrode 1 has an electrically active tip 2 as well as a second electric stimulation pole in the form of a ring electrode 2 directly behind the tip 2. The screw typically has 3-4 (2-20) windings. By screwing into the nerve plexus 3, the electrode ring directly behind the screw comes in contact with the epicardial nerve plexus 3, thus ensuring bipolar electric stimulation of the nerve plexus 3 (see FIG. 19).

As an alternative, the neurostimulation electrode may be made of a flexible electrode catheter 1, along the length of which are attached one or more circular stimulation poles 2 (electrode length 2-5 mm, interelectrode spacing 2-10 mm). Between the stimulation poles 2 there are inlet openings 17 which communicate with one another through a connecting lumen 18 and to which a vacuum can be applied. The inlet openings 17 may also have so-called lips 19 to facilitate contact with epicardium. By applying a vacuum, the catheter is secured on the epicardial nerve plexus 3 (see FIG. 20).

In all these embodiments, the stimulation poles are connected to a stimulation unit by electrically conducting wires. The stimulation unit consists of a pulse generating unit and a starter unit.

The neurostimulation unit also includes a pulse generating unit 21, the operation of which is described in greater detail below with reference to FIG. 21. The pulse generating unit 21 is preferably a voltage generator capable of generating electric stimulation pulses. The pulse duration may be between 0 and 20 ms (typically 0.05 to 5 ms) and the stimulation frequency may be between 0 and 1000 Hz (typically 2-100 Hz). The pulse shape may be monophasic, biphasic or triphasic. The stimulation voltage may be between 1 and 100 V. Generally, continuous epicardial/extravascular stimulation of the nerve plexus is provided. In the individual case, however, an intermittent pulsed neurostimulation in the atrial/ventricular refractory time may be necessary to prevent atrial/ventricular electric myocardial stimulation of the myocardium beneath the nerve plexus. Therefore, short bursts of high-frequency electric stimuli (frequency 1-100 Hz, typically 200 Hz) are triggered so that the stimuli are coupled to the atrial P wave or ventricular R wave. The coupling interval is typically 20 ms, but it may assume any value between 0 ms and 100 ms. Atrial or ventricular myocardial depolarization as well as the subsequent refractory phase of the heart are detected by a first detection unit 28, which is connected to the control unit 19. This is accomplished either via the neurostimulation poles 2, which may serve as sensing electrodes, or by means of endocardially or epicardially positioned atrial and/or ventricular sensing electrodes. Alternatively, the pulsed neurostimulation may also be performed triggered at the P wave or the R wave in the surface ECG. The atrial/ventricular sensing signals are also needed to adapt the intensity of the neurostimuli to the particular atrial/ventricular frequency. The atrial/ventricular signals are therefore transmitted to the control device 19 which actuates and/or triggers the pulse generating unit as a function of the state of the first detection device.

The control unit 19 may also be connected to a second detection unit 19.2 which is in turn connected to one or more measurement probes. The second detection unit 30 is used here to detect the cardiac output. In other variants of this invention, it may also be used to detect other biological or mechanical measured variables such as heart rate, blood pressure, oxygen partial pressure, repolarization times, changes in the excitation regeneration of the heart, body temperature and mechanical movements such as changes in the positions of the arm of a surgical robot or of the surgeon. A starting unit of the control unit 19 which responds to the detection variables starts operation of the pulse generating unit 21 as soon as the particular measured value is above or below certain limit values. According to a modification of the starter unit, the surgeon, by using a (foot) switch 26, controls the beginning, duration and intensity of the neurostimulation on the basis of the movement of the heart and/or the immobilization of the heart which is considered to be necessary. Thus, like a gas pedal in an automobile, by applying a varying pressure with the foot which is exerted on a foot switch, the extent of the neurostimulation and bradycardization is adapted to the technical surgical needs at any time. Depending on the basic heart pump function, despite the increase in stroke volume, beyond a certain extent bradycardization is associated with a reduction in cardiac output. To prevent a critical reduction in circulation during neurostimulation, biological parameters such as cardiac output, arterial oxygen partial pressure or arterial/central venous pressure are displayed to the surgeon so that he can then adapt the extent and duration of bradycardization to the hemodynamic needs of the patient. In one variant of this invention, the neurostimulation intensity is automatically reduced by the control unit 19 if the cardiac output falls below a lower limit.

In addition to the present invention, which describes exclusive parasympathetic neurostimulation to facilitate surgery on a beating heart, according to one variant of the present invention, a neurostimulation unit is combined with systems for local mechanical stabilization/immobilization of the myocardial area in the immediate vicinity of a coronary vessel. The electric immobilization of the heart achieved by neurostimulation is synergistic with local immobilization through the use of stabilizers.

In another preferred embodiment, the neurostimulation unit is combined with a pump unit in the form of an intravascular or paracardial blood pump or an extracorporeal blood pump as a component of a motion reducing device. The neurostimulation unit 20, which comprises the stimulation electrode 1 and the pulse generating unit 21 is connected to a control unit 19. The same applies for the motion reducing device 22 which includes the blood pump 22 and its pump control 24. The blood pump 22 should first of all maintain the cardiac output which is reduced due to the bradycardia, and, furthermore, relieve the stroke volume of a single heartbeat, which is elevated due to the bradycardia and results in an increased wall movement during a single contraction. To this end, in a first operating mode, the control unit 19 automatically increases the cardiac output provided by the pump as soon as the heart rate is reduced due to neurostimulation. In other words, the control unit 19 controls the movement reducing device 22 in this operating mode as a function of the type of stimulation and in particular the intensity of the stimulation. Furthermore, according to a modification of the combined pump and neurostimulation system, in a second operating mode, separate manual control of the neurostimulator 20 and the pump 23 is possible. Thus, the surgeon is able to select, depending on the needs of the operation and by means of two foot pedals, for example, the extent of the mechanical relief to be provided by the blood pump 23 and/or of the electric immobilization due to neurostimulation. In this case, the minimum required degree of pump activity and/or the maximum available degree of bradycardia are determined by the programmed lower limits of the cardiac output. Thus, for example, the degree of bradycardia cannot be increased further due to neurostimulation if the cardiac output falls below the minimum limit while at maximum pump activity. Such a combination of neurostimulation with a paracardial pump is also suitable in special cases for the heart supportive therapy over a period of weeks or even months during which a paracardial pump 23 is implanted to relieve the volume burden on the heart and allow it to recover. Since lowering the heart rate contributes to a reduction in myocardial oxygen consumption, the proposed invention can contribute toward recovery of the myocardial tissue by lowering the heart rate. In this case, the neurostimulation electrodes 1 are anchored (semi)permanently in the nerve plexus 3, e.g., by screw or clamping mechanisms at the tip of the electrodes 1. The electrodes are then connected to a (chronically) implantable neurostimulator that contains a battery and appropriate software components for cardiac neurostimulation. Such a device is described in U.S. patent 2002/0026222 A1, for example. In advantageous variants of this invention, a switching device 19.1 is connected to the control unit 19 for switching between the first and the second operating mode.

In a special embodiment of the combination of neurostimulation with intra-/paracardial blood pump 23, the outlet opening of the intracardiac pump 23 which is situated in the proximal pulmonary artery (arteria pulmonalis communis) is lengthened by adding a neurostimulation electrode 1. The neurostimulation electrode is to be guided through a separate lumen to the intracardiac pump 23 into the right (left) pulmonary artery, where it electrically transvascularly stimulates parasympathetic nerves 3 that innervate the sinus node and AV node (see FIG. 21). As an alternative, a stimulation electrode that is fixedly connected to the blood pump may also be positioned in the right (left) pulmonary artery as an extension of the blood pump.

In a special embodiment, the outlet opening of the blood pump itself has a stimulation electrode 2. The outlet opening of the blood pump 23 is advanced into the right (left) pulmonary artery and is reversibly secured in the vessel by inflation of a balloon 25. The balloon 25 does thereby not occlude the vessel but instead has outlet openings for maintaining the pulmonary artery flow. The balloon 25, at its circumference, also has two stimulation poles 2, contacting with the pulmonary arterial wall as a result of inflation of the balloon and by which the nerves 3 situated on the outside of the pulmonary artery may be electrically stimulated transvascularly. To ensure perfusion of the contralateral pulmonary artery as well when advancing the outlet opening into one of the two pulmonary arteries, the outlet tube of the blood pump 23 has another lateral outlet opening in the area of the branching point of the contralateral pulmonary artery (see FIG. 22).

The control loop for a combined electric and mechanical immobilization is described below as an example (see FIGS. 21 and 23). With a first input device 26 in the form of a (foot) switch connected to the control unit 19, the surgeon selects the extent of the desired electric immobilization (=heart rate retardation). According to previously compiled dose-effect curves, a neurostimulation intensity is provided to achieve the heart rate as the target variable. This course of the stimulation dose and immobilization effect is stored in a suitably representative patient data record in a second input device 27 that is connected to the control unit 19. The control unit influences the neurostimulation unit 20 in the manner described below depending on the patient data record. In parallel with this, the heart rhythm (sinus rhythm or atrial fibrillation, for example) is verified and the proper epicardial neurostimulation site is selected (sinus rhythm: ventral right atrial plexus; atrial fibrillations: inferior interatrial plexus). The (change in) heart rate achieved is measured by a heart rate detection unit 28 that is connected to the control unit 19. If the actual heart rate differs from the target rate which is preselected via a third input device 29 connected to the control unit 19, the neurostimulation intensity is automatically increased/reduced via a feedback loop. Such deviations in an individual patient-specific dose-effect curve are detected and transmitted as correction factors to the regulating unit of the neurostimulation unit so that an individual dose-effect curve is created for the patient. The stroke volume or the cardiac output, respectively, is measured via a cardiac output detection unit 30 in the form of a hemodynamic detection unit connected to the control unit 19 and this information is transmitted together with the heart rate to a comparator unit 19.1 of the control unit 19. Said comparator unit compares the actual cardiac output (HZV) with the setpoint HZV preselected by a fourth input unit 31 that is connected to the control unit 19. Since cardiac output may decline as a result of the retardation in heart rate, the cardiac output delivered through a paracardial pump is increased in a controlled way by the control unit 19 when the cardiac output falls below a critical level. Conversely, the pump HZV is automatically reduced by the control unit 19 when the heart rate increases. If mechanical immobilization is additionally desired by the surgeon at a given extent of electric immobilization (bradycardia), then the HZV provided by the paracardial pump 23 may also be increased directly by the surgeon through appropriate action on the control unit 19 in order to relieve the volume burden on the heart. The definition of the setpoint HZV limits also includes patient-specific parameters (e.g., co-morbidity such as cerebral vascular constriction, renal function, basal cardiac pump capacity, etc.). Such a system allows the surgeon the necessary free choice of the degree and duration of the electric immobilization without compromising the patient due to a consecutively reduced cardiac output.

In other words, the control unit 19 influences both the operating state of the neurostimulation unit 20 as well as of the movement reducing device 22 in accordance with the specifications of the first through fourth input devices 26, 27, 29 and 31 as well as the operating states of the heart rate detection device 28 and the cardiac output detection device 30.

In one exemplary embodiment with exclusive electric immobilization of the heart by cardiac neurostimulation, when the cardiac output drops below certain critical lower limits, the reduction in heart rate due to neurostimulation is ramped down (see FIG. 24).

1. A device for temporarily reducing the movement of the heart during surgery, comprising:
a neurostimulation device for stimulating nerves that slow down the heart rate, having at least one electrode device with at least one stimulation pole and
a control unit being connected to the neurostimulation device having a first input device for preselecting a degree of electric immobilization of the heart and being arranged for patient specifically influencing the operation mode of the neurostimulation device as a function of the preselected degree of electric immobilization, wherein the first input device is arranged for variably preselecting the degree of electric immobilization during operation.

2. The device according to claim 1, wherein the stimulation pole of the electrode device has an effective stimulation area of 1 to 100 mm².

3. The device according to claim 1 wherein the electrode device comprises at least two stimulation poles for bipolar stimulation that are arranged spatially separate.

4. The device according to claim 3, wherein the stimulation poles have a distance between each other that is between about 2 and about 10 mm.

5. The device according to claim 1, wherein the electrode device inserts into a nerve plexus.

6. The device according to claim 1, wherein the electrode device comprises at least one locking device for securing the electrode device on a location selected from the group consisting of a nerve plexus, a blood vessel, and a combination thereof.

7. The device according to claim 6, wherein the at least one locking device comprises at least one fastening device with at least two arms for securely clamping the electrode device and wherein at least one stimulation pole is arranged in an area of a free end of a forceps arm.

8. The device according to claim 6, wherein the locking device comprises at least one suction device having at least one suction opening for fastening the electrode device to human tissue by employment of a vacuum.

9. The device according to claim 8, wherein the stimulation pole is situated in an area of the suction opening.

10. The device according to claim 6, wherein the locking device comprises at least one supply channel for tissue adhesive with at least one mouth opening for securing the electrode device with the adhesive in the area of the mouth opening.

11. The device according to claim 10, wherein the stimulation pole is situated in the area of the mouth opening.

12. The device according to claim 1, wherein the electrode device is a screw electrode.

13. The device according to claim 1, wherein the electrode device comprises a shielding device that is provided for the stimulation pole to prevent unwanted stimulation of cardiac tissue.

14. The device according to claim 1, wherein the neurostimulation device comprises a pulse generating unit that is connected to the electrode device and is also connected to the control unit for triggering purposes.

15. The device according to claim 14, wherein the pulse generating unit generates pulses that have a characteristic selecting from the group consisting of a duration between 0 and 20 ms, a stimulation frequency between 0 and 1000 Hz, a stimulation voltage between 1 and 100 V, and any combinations thereof.

16. The device according to claim 14 wherein the pulse generating unit provides continuous stimulation.

17. The device according to claim 14 wherein the pulse generating unit provides intermittent stimulation, generating short bursts of high-frequency pulses.

18. The device according to claim 17, further comprising a first detection unit that is connected to the control unit for detecting a refractory phase of the heart, wherein the control unit operates the pulse generating unit as a function of a state of the first detection unit.

19. The device according to claim 18, wherein the first detection unit comprises at least one sensing electrode that is formed by the electrode device.

20. The device according to one of the claim 1, further comprising at least one second detection unit connected to the control unit, for detecting at least one biological or human measured variable, wherein the control unit influences the neurostimulation device as a function of a state of the second detection unit.

21. The device according to claim 1, further comprising a movement reducing device that is connected to the control unit, wherein the control unit influences an operating state of the movement reducing device.

22. The device according to claim 21, wherein the control unit is in an operating mode selected from the group consisting of a first operating mode, a second operating mode, and a combination thereof, and wherein: the first operating mode, the control unit influences an operating state of the movement reducing device as a function of an operating state of the neurostimulation device and, and in a second operating mode, the control unit separately influences the operating state of the movement reducing device and the neurostimulation device.

23. The device according to claim 22, wherein the control unit comprises a switching device for switching between the first operating mode and the second operating mode.

24. The device according to claim 21 wherein the movement reducing device comprises a device selected from the group consisting of a pump device for supporting cardiac function, a stabilization device for stabilizing the cardiac wall, and a combination thereof.

25. The device according to claim 21, wherein the control unit controls the operating state of the movement reducing device as a function of information selected from the group consisting of a type of stimulation of the neurostimulation device, a stimulation intensity of the neurostimulation device, and a combination thereof.

26. The device according to claim 21, further comprising a second input device that is connected to the control unit for storing at least one patient-specific data record that is representative of a course of a stimulation dose and a immobilization effect, and wherein the control unit influences the neurostimulation device as a function of the patient data record.

27. The device according to claim 21, further comprising a heart rate detection device that is connected to the control unit for detecting a heart rate signal that is representative of the actual heart rate,
wherein the control unit influences a device selected from the group consisting of the neurostimulation device the movement reducing device and a combination thereof, as a function of a state of the heart rate detection device.

28. The device according to claim 27, further comprising a third input device that is connected to the control unit for input of a setpoint heart rate,
wherein the control unit influences a device selected from the group consisting of the neurostimulation device the movement reducing device and a combination thereof, as a function of the state of the heart rate detection device and the third input device.

29. The device according to claim 21, further comprising a cardiac output detection device that is connected to the control unit for detecting a cardiac output signal that is representative of the actual cardiac output,
wherein the control unit influences a device selected from the group consisting of the neurostimulation device, the movement reducing device, and a combination thereof, as a function of the state of the cardiac output detection device.

30. The device according to claim 29, further comprising a fourth input device that is connected to the control unit for input of a setpoint cardiac output,
wherein the control device influences a device selected from the group consisting of the neurostimulation device the movement reducing device, and a combination thereof, as a function of the state of the cardiac output detection device and the fourth input device.

31. The device according to claim 21, wherein the movement reducing device comprises a pump unit for supporting heart function, and wherein the electrode device of the neurostimulation device is situated on the pump unit.

32. The device according to claim 31, wherein the stimulation pole is situated on the pump unit.

33. The device according to claim 1, wherein the stimulation pole of the electrode device has an effective stimulation area of 4 to 9 mm².

34. The device according to claim 14, wherein the pulse generating unit generates pulses that have a characteristic selected from the group consisting of a duration between 0.05 to 5 ms, a stimulation frequency between 2 to 100 Hz, a stimulation voltage between 1 and 100 V, and any combinations thereof.

35. A method for temporarily reducing the movement of a patient's heart during surgery, comprising:
pre-selecting a degree of electric immobilization of the heart; and
providing stimulation to nerves that slow down a heart rate,
wherein said stimulation is patient specifically influenced as a function of the pre-selected degree of electric immobilization, and the pre-selected degree of electric immobilization is variable during operation.

36. The method according to claim 35, wherein the stimulation to the nerves is bipolar stimulation.

37. The method according to claim 35, wherein the stimulation to the nerves is provided to a nerve plexus of the nerves.

38. The method according to claim 37, further comprising inserting an electrode device into the nerve plexus.

39. The method according to claim 35, further comprising securing an electrode device on a location selected from the group consisting of a nerve plexus, a blood vessel, and a combination thereof, wherein the stimulation to the nerves is provided by the electrode device.

40. The method according to claim 39, wherein the electrode device is secured on the location by a technique selected from the group consisting of applying a clamping force, applying a suction force, applying a tissue adhesive, applying a screw connection, and any combinations thereof.

41. The method according to claim 35, further comprising shielding tissue surrounding a location of the stimulation to the nerves to prevent unwanted stimulation of cardiac tissue.

42. The method according to claim 35, wherein the stimulation to the nerves is provided by stimulation pulses, wherein the stimulation pulses have a characteristic selected from the group consisting of a duration between 0 and 20 ms, a stimulation frequency between 0 and 1000 Hz, a stimulation voltage between 1 and 100 V, and any combinations thereof.

43. The method according to claim 35, wherein the stimulation to the nerves is provided by continuous stimulation.

44. The method according to claim 35, wherein the stimulation to the nerves is provided by intermittent stimulation, wherein the intermittent stimulation includes generating short bursts of high-frequency pulses.

45. The method according to claim 35, further comprising detecting a refractory phase of the heart, wherein the stimulation to the nerves is provided as a function of the detected refractory phase of the heart.

46. The method according to claim 35, further comprising detecting at least one biological or human measured variable, wherein the stimulation to the nerves is provided as a function of the detected biological or human measured variable.

47. The method according to claim 35, further comprising:
providing a movement reducing device; and
providing an operating mode selected from the group consisting of a first operating mode, a second operating mode, and a combination thereof, wherein in the first operating mode, an operating state of the movement reducing device is influenced as a function of the stimulation to the nerves, and in the second operating mode, an operating state of the movement reducing device is influenced separately from the stimulation to the nerves.

48. The method according to claim 47, further comprising switching between the first operating mode and the second operating mode.

49. The method according to claim 35, further comprising providing a movement reducing device, wherein the movement reducing device is selected from the group consisting of a pump device for supporting cardiac function, a stabilization device for stabilizing the cardiac wall, and a combination thereof.

50. The method according to claim 49, further comprising controlling an operating state of the movement reducing device as a function of information selected from the group consisting of a type of the stimulation to the nerves, a stimulation intensity of the stimulation to the nerves, and a combination thereof.

51. The method according to claim 35, wherein the stimulation to the nerves is provided as a function of at least one patient-specific patient data record, wherein the patient-specific patient data record is representative of a patient-specific course of a stimulation dose and an immobilization effect.

52. The method according to claim 35, further comprising:
detecting a heart rate signal representative of the patient's actual heart rate; and
controlling an application as a function of said detected heart rate signal, wherein the application is selected from the group consisting of the stimulation to the nerves, operation of a movement reducing device, and a combination thereof.

53. The method according to claim 53, further comprising:
selecting a setpoint heart rate; and
controlling the application as a function of said selected setpoint heart rate.

54. The device according to claim 21, further comprising a device for detecting a cardiac output signal representative of the patient's actual cardiac output;
wherein an application is controlled as a function of said detected cardiac output signal, and wherein the application is selected from the group consisting of stimulation to the nerves that slow down the heart rate, operation of the movement reducing device, and a combination thereof.

55. The method according to claim 35, further comprising:
selecting a setpoint cardiac output; and
controlling an application as a function of said selected setpoint cardiac output, wherein the application is selected from the group consisting of the stimulation to the nerves, operation of a movement reducing device, and a combination thereof.
